(19) United States
(12) Patent Application Publication
(10) Pub. No.: US 2025/0352027 A1
(43) Pub. Date: Nov. 20, 2025

(54) MEDICAL SUPPORT DEVICE, ENDOSCOPE, MEDICAL SUPPORT METHOD, AND PROGRAM

(71) Applicant: FUJIFILM Corporation, Tokyo (JP)

(72) Inventor: Masaaki OOSAKE, Kanagawa (JP)

(73) Assignee: FUJIFILM Corporation, Tokyo (JP)

(21) Appl. No.: 19/284,644

(22) Filed: Jul. 29, 2025

Related U.S. Application Data
(63) Continuation of application No. PCT/JP2024/002653, filed on Jan. 29, 2024.

(30) Foreign Application Priority Data
Feb. 16, 2023 (JP) 2023-022704

(51) Int. Cl. A61B 1/00; G06T 7/00; G06T 7/62

(52) U.S. Cl. CPC A61B 1/000094; A61B 1/000096; A61B 1/0005; G06T 7/0012; G06T 7/62; G06T 2207/10068; G06T 2207/20; G06T 2207/30096

(57) ABSTRACT

A medical support device includes a processor. The processor is configured to recognize, based on a medical image in which a plurality of observation target regions appear, positions of the plurality of observation target regions in the medical image, determine a priority of the plurality of observation target regions, based on the positions, measure sizes of the plurality of observation target regions, and output the sizes in accordance with the priority.

### CROSS-REFERENCE TO RELATED APPLICATIONS

This application is a continuation application of International Application No. PCT/JP2024/002653, filed Jan. 29, 2024, the disclosure of which is incorporated herein by reference in its entirety. Further, this application claims priority from Japanese Patent Application No. 2023-022704, filed Feb. 16, 2023, the disclosure of which is incorporated herein by reference in its entirety.

### BACKGROUND

### 1. Technical Field

The technology of the present disclosure relates to a medical support device, an endoscope, a medical support method, and a program.

### 2. Related Art

JP2021-178110A discloses an information processing apparatus located between an imaging control device and an image management device in communication. The information processing apparatus described in JP2021-178110A includes an abnormality detection means for detecting an abnormality from a medical image received from the imaging control device, and a control means for determining, based on a detection result obtained by the abnormality detection means, the content of the medical image or the detection result to be transmitted or a destination to which the medical image or the detection result is to be transmitted.

In the information processing apparatus described in JP2021-178110A, the abnormality detection means generates a lesion name, a lesion position, a lesion size, a lesion category, a grade of malignancy, or a malignancy map from the medical image. In the information processing apparatus described in JP2021-178110A, furthermore, the control means assigns a priority level for displaying the medical image to the medical image, based on the detection result, and transmits the priority level to the image management device in association with the medical image.

WO2020/188682A discloses a diagnosis support apparatus. The diagnosis support apparatus described in WO2020/188682A has an abnormal symptom identifying unit, a lesion extraction function unit, and a function control unit.

The abnormal symptom identifying unit is configured to perform a process for identifying one abnormal symptom appearing in a diagnosis target organ of a subject, based on at least one of physical information or an endoscopic image, the physical information including one or more pieces of information from which a state of the diagnosis target organ can be estimated, the endoscopic image being obtained by imaging the diagnosis target organ. The lesion extraction function unit is configured to have a plurality of different lesion extraction units specialized for respective abnormal symptoms that may appear in the diagnosis target organ, as lesion extraction processing for extracting a candidate lesion region from the endoscopic image. The lesion extraction function control unit is configured to perform a process for selecting one lesion extraction unit corresponding to the one abnormal symptom identified by the abnormal symptom identifying unit from among the plurality of lesion extraction units, and control the lesion extraction function unit to cause the one lesion extraction unit to perform the lesion extraction processing.

The diagnosis support apparatus described in WO2020/188682A further has a display control unit. The display control unit is configured to perform a process for causing a display device to display both the endoscopic image and information indicating a position of the candidate lesion region extracted by the one lesion extraction unit.

### SUMMARY

An embodiment according to the technology of the present disclosure provides a medical support device, an endoscope, a medical support method, and a program that enable a user or the like to grasp the size of an observation target region expected to be of high interest to the user or the like when a plurality of observation target regions appear in a medical image.

A first aspect according to the technology of the present disclosure is a medical support device including a processor, the processor being configured to recognize, based on a medical image in which a plurality of observation target regions appear, positions of the plurality of observation target regions in the medical image, determine a priority of the plurality of observation target regions, based on the positions, measure sizes of the plurality of observation target regions, and output the sizes in accordance with the priority.

A second aspect according to the technology of the present disclosure is the medical support device according to the first aspect, in which the processor is configured to output the sizes of the observation target regions one at a time in an order based on the priority.

A third aspect according to the technology of the present disclosure is the medical support device according to the second aspect, in which each of the sizes of the observation target regions is output each time an instruction is given.

A fourth aspect according to the technology of the present disclosure is the medical support device according to any one of the first to third aspects, in which the sizes are output by displaying the sizes on a screen.

A fifth aspect according to the technology of the present disclosure is the medical support device according to the fourth aspect, in which the sizes are displayed on the screen in a display format corresponding to the priority.

A sixth aspect according to the technology of the present disclosure is the medical support device according to the fourth aspect or the fifth aspect, in which the screen displays the medical image, and the sizes are displayed in the medical image.

A seventh aspect according to the technology of the present disclosure is the medical support device according to any one of the fourth to sixth aspects, in which the screen displays the medical image, and region identification information is displayed in the medical image, the region identification information being information that allows an observation target region corresponding to a size that has been output among the sizes to be identified from among the observation target regions.

An eighth aspect according to the technology of the present disclosure is the medical support device according to any one of the fourth to seventh aspects, in which the screen includes a first display region and a second display region, the first display region displays the medical image, and the second display region displays a map indicating a distribution of the positions of the observation target regions, and region identification information is displayed on the map, the region identification information being information that allows an observation target region corresponding to a size that has been output among the sizes to be identified from among the observation target regions.

A ninth aspect according to the technology of the present disclosure is the medical support device according to any one of the fourth to eighth aspects, in which a size to be displayed on the screen among the sizes is switched in accordance with the priority.

A tenth aspect according to the technology of the present disclosure is the medical support device according to the ninth aspect, in which the size to be displayed on the screen is switched each time an instruction is given.

An eleventh aspect according to the technology of the present disclosure is the medical support device according to any one of the first to tenth aspects, in which the positions are recognized by a method using AI, and the priority is determined based on a degree of certainty obtained from the AI.

A twelfth aspect according to the technology of the present disclosure is the medical support device according to any one of the first to eleventh aspects, in which the priority is higher for a position closer to a center of the medical image among the positions.

A thirteenth aspect according to the technology of the present disclosure is the medical support device according to any one of the first to twelfth aspects, in which the processor is configured to acquire depths of the plurality of observation target regions, and the priority is determined based on the positions and the depths.

A fourteenth aspect according to the technology of the present disclosure is the medical support device according to any one of the first to thirteenth aspects, in which the processor is configured to recognize types of the observation target regions, based on the medical image, and the priority is determined based on the positions and the types.

A fifteenth aspect according to the technology of the present disclosure is the medical support device according to any one of the first to fourteenth aspects, in which the processor is configured to measure the sizes in accordance with the priority.

A sixteenth aspect according to the technology of the present disclosure is the medical support device according to any one of the first to fifteenth aspects, in which the medical image is an endoscopic image obtained by imaging with an endoscope.

A seventeenth aspect according to the technology of the present disclosure is the medical support device according to any one of the first to sixteenth aspects, in which the observation target regions are lesions.

An eighteenth aspect according to the technology of the present disclosure is an endoscope including the medical support device according to any one of the first to seventeenth aspects, and a module to be inserted into a body including the observation target regions to acquire the medical image by imaging the observation target regions.

A nineteenth aspect according to the technology of the present disclosure is a medical support method including recognizing, based on a medical image in which a plurality of observation target regions appear, positions of the plurality of observation target regions in the medical image; determining a priority of the plurality of observation target regions, based on the positions; measuring sizes of the plurality of observation target regions; and outputting the sizes in accordance with the priority.

A twentieth aspect according to the technology of the present disclosure is a program for causing a computer to execute a medical support process including recognizing, based on a medical image in which a plurality of observation target regions appear, positions of the plurality of observation target regions in the medical image; determining a priority of the plurality of observation target regions, based on the positions; measuring sizes of the plurality of observation target regions; and outputting the sizes in accordance with the priority.

### BRIEF DESCRIPTION OF THE DRAWINGS

Exemplary embodiments according to the technique of the present disclosure will be described in detail based on the following figures, wherein:

FIG. 1 is a conceptual diagram illustrating an example of an aspect in which an endoscope system is used;

FIG. 2 is a conceptual diagram illustrating an example overall configuration of an endoscope system;

FIG. 3 is a block diagram illustrating an example hardware configuration of an electric system of the endoscope system;

FIG. 4 is a block diagram illustrating an example of functions of main components of a processor included in the endoscope, and an example of information stored in an NVM;

FIG. 5 is a conceptual diagram illustrating an example of the content of a process performed by a recognition unit and a control unit;

FIG. 6 is a conceptual diagram illustrating an example of the content of a process performed by a determination unit;

FIG. 7 is a conceptual diagram illustrating an example of the content of a process performed by a measurement unit;

FIG. 8 is a conceptual diagram illustrating an example of an aspect in which an endoscopic image is displayed in a first display region and a size is displayed on a map in a second display region;

FIG. 9 is a flowchart illustrating an example of the flow of a medical support process;

FIG. 10 is a conceptual diagram illustrating an example aspect in which the content displayed on the map is switched in accordance with an instruction received by a reception device;

FIG. 11 is a conceptual diagram illustrating a first modification of the content of the process performed by the determination unit;

FIG. 12 is a conceptual diagram illustrating a second modification of the content of the process performed by the determination unit;

FIG. 13 is a conceptual diagram illustrating an example aspect in which a segmentation image displayed on the map is enclosed by a circumscribed rectangular frame;

FIG. 14 is a conceptual diagram illustrating an example aspect in which sizes and text information are displayed outside the map in a pop-up manner from within the map and are displayed on a screen in display sizes based on the priority;

FIG. 15 is a conceptual diagram illustrating an example aspect in which the sizes and the text information are displayed outside the endoscopic image in a pop-up manner from within the endoscopic image and are displayed on a screen in display sizes based on the priority;

FIG. 16 is a conceptual diagram illustrating an example aspect in which a lesion appearing in the endoscopic image is enclosed by a circumscribed rectangular frame; and

FIG. 17 is a conceptual diagram illustrating examples of a size output destination.

### DETAILED DESCRIPTION

An example of an embodiment of a medical support device, an endoscope, a medical support method, and a program according to the technology of the present disclosure will be described hereinafter with reference to the accompanying drawings.

First, terms used in the following description will be described.

CPU is an abbreviation for “Central Processing Unit”. GPU is an abbreviation for “Graphics Processing Unit”. RAM is an abbreviation for “Random Access Memory”. NVM is an abbreviation for “Non-volatile memory”. EEPROM is an abbreviation for “Electrically Erasable Programmable Read-Only Memory”. ASIC is an abbreviation for “Application Specific Integrated Circuit”. PLD is an abbreviation for “Programmable Logic Device”. FPGA is an abbreviation for “Field-Programmable Gate Array”. SoC is an abbreviation for “System-on-a-chip”. SSD is an abbreviation for “Solid State Drive”. USB is an abbreviation for “Universal Serial Bus”. HDD is an abbreviation for “Hard Disk Drive”. EL is an abbreviation for “Electro-Luminescence”. CMOS is an abbreviation for “Complementary Metal Oxide Semiconductor”. CCD is an abbreviation for “Charge Coupled Device”. AI is an abbreviation for “Artificial Intelligence”. BLI is an abbreviation for “Blue Light Imaging”. LCI is an abbreviation for “Linked Color Imaging”. I/F is an abbreviation for “Interface”. SSL is an abbreviation for “Sessile Serrated Lesion”. NP refers to “Neoplastic Polyp”. HP refers to “Hyperplastic Polyp”.

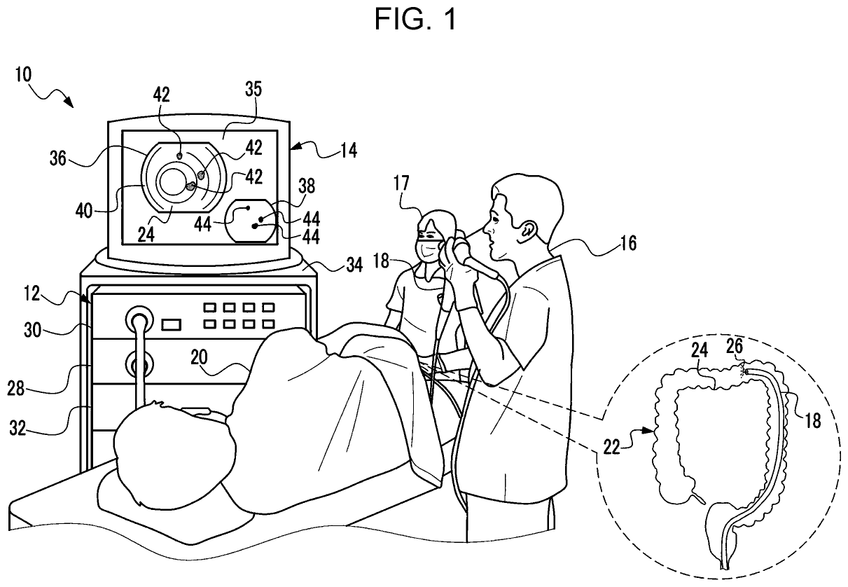

As an example, as illustrated in FIG. 1, an endoscope system 10 includes an endoscope 12 and a display device 14. The endoscope 12 is used by a doctor 16 in an endoscopic examination. The endoscopic examination is assisted by staff such as a nurse 17. In the present embodiment, the endoscope 12 is an example of an “endoscope” according to the technology of the present disclosure.

The endoscope 12 is connected to a communication device (not illustrated) in a communicable manner, and information obtained by the endoscope 12 is transmitted to the communication device. An example of the communication device is a server and/or a client terminal (for example, a personal computer and/or a tablet terminal) that manages various kinds of information such as electronic medical records. The communication device receives the information transmitted from the endoscope 12 and executes a process using the received information (for example, a process of storing the information in an electronic medical record or the like).

The endoscope 12 includes an endoscope main body 18. The endoscope 12 is an apparatus for performing medical care for a large intestine 22 included in the body of a subject 20 (for example, a patient) using the endoscope main body 18. In the present embodiment, the large intestine 22 is a target to be observed by the doctor 16.

The endoscope main body 18 is inserted into the large intestine 22 of the subject 20. The endoscope 12 causes the endoscope main body 18 inserted into the large intestine 22 of the subject 20 to perform imaging of the inside of the large intestine 22 in the body of the subject 20, and performs various medical treatments on the large intestine 22 as necessary.

The endoscope 12 performs imaging of the inside of the large intestine 22 of the subject 20 to acquire an image indicating the state of the inside of the body, and outputs the acquired image. In the present embodiment, the endoscope 12 is an endoscope having an optical imaging function of capturing an image of reflected light obtained by irradiating the inside of the large intestine 22 with light 26 and reflecting the light 26 from an intestinal wall 24 of the large intestine 22.

While the endoscopic examination of the large intestine 22 is illustrated here, this is merely an example, and the technology of the present disclosure is also applicable to an endoscopic examination of a luminal organ such as the esophagus, the stomach, the duodenum, or the trachea.

The endoscope 12 includes a control device 28, a light source device 30, and an image processing device 32. The control device 28, the light source device 30, and the image processing device 32 are installed in a cart 34. The cart 34 is provided with a plurality of shelves along the vertical direction, and the image processing device 32, the control device 28, and the light source device 30 are installed on the shelves from bottom to top. The display device 14 is installed on top of the cart 34.

The control device 28 controls the entire endoscope 12. The image processing device 32 performs various kinds of image processing on an image obtained by imaging of the intestinal wall 24 using the endoscope main body 18, under the control of the control device 28.

The display device 14 displays various kinds of information including images. An example of the display device 14 is a liquid crystal display or an EL display. A tablet terminal with a display may be used instead of or together with the display device 14.

The display device 14 displays a screen 35. The screen 35 includes a plurality of display regions. The plurality of display regions are arranged side by side on the screen 35. In the example illustrated in FIG. 1, a first display region 36 and a second display region 38 are illustrated as an example of the plurality of display regions. The size of the first display region 36 is larger than the size of the second display region 38. The first display region 36 is used as a main display region, and the second display region 38 is used as a sub-display region. In the present embodiment, the screen 35 is an example of a “screen” according to the technology of the present disclosure, the first display region 36 is an example of a “first display region” according to the technology of the present disclosure, and the second display region 38 is an example of a “second display region” according to the technology of the present disclosure.

The first display region 36 displays an endoscopic image 40. The endoscopic image 40 is an image acquired by imaging of the intestinal wall 24 in the large intestine 22 of the subject 20 using the endoscope main body 18. In the example illustrated in FIG. 1, an image in which the intestinal wall 24 appears is illustrated as an example of the endoscopic image 40. Further, the intestinal wall 24 appearing in the endoscopic image 40 includes a plurality of lesions 42 (for example, in the example illustrated in FIG. 1, three lesions 42) as a plurality of regions of interest (that is, a plurality of observation target regions) to be gazed at by the doctor 16, and the doctor 16 can visually recognize the state of the intestinal wall 24 including the plurality of lesions 42 through the endoscopic image 40. There are various types of lesions 42, and the types of lesions 42 include, for example, a neoplastic polyp (for example, an NP or an SSL belonging to the NP) and a non-neoplastic polyp (for example, an HP).

In the present embodiment, the endoscopic image 40 is an example of a “medical image” and an “endoscopic image” according to the technology of the present disclosure. In the present embodiment, the lesions 42 are an example of “observation target regions” and “lesions” according to the technology of the present disclosure. While the lesions 42 are illustrated here, the technology of the present disclosure is not limited to this. The plurality of regions of interest (that is, the plurality of observation target regions) to be gazed at by the doctor 16 may be a plurality of organs (for example, a bile duct opening and a pancreatic duct opening included in the duodenal papilla), a plurality of marked regions, artificial treatment tools (for example, artificial clips), a plurality of treated regions (for example, a plurality of regions with traces of removal of polyps or the like), or the like. The plurality of regions of interest (that is, the plurality of observation target regions) to be gazed at by the doctor 16 may be a combination of a plurality of elements among at least one lesion 42, at least one organ, at least one marked region, at least one artificial treatment tool, and at least one treated region.

The first display region 36 displays a moving image. The endoscopic image 40 displayed in the first display region 36 is one frame included in a moving image configured to include a plurality of frames along a time series. That is, the first display region 36 displays endoscopic images 40 of a plurality of frames at a specified frame rate (for example, 30 frames/second, 60 frames/second, or the like).

An example of the moving image to be displayed in the first display region 36 is a live-view moving image. The live-view moving image is merely an example, and the moving image may be a moving image that is temporarily stored in a memory or the like before being displayed, like a post-view moving image. Alternatively, each frame included in a recording moving image stored in the memory or the like may be reproduced and displayed in the first display region 36 as the endoscopic image 40.

On the screen 35, the second display region 38 is adjacent to the first display region 36 and is displayed in a lower right portion of the screen 35 when viewed from the front. The second display region 38 may be displayed at any position within the screen 35 of the display device 14, but is preferably displayed at a position that enables comparison with the endoscopic image 40. The second display region 38 displays a plurality of segmentation images 44. The segmentation images 44 are image regions for identifying the positions of the lesions 42, which are recognized by performing an object recognition process using an AI-based segmentation method on the endoscopic image 40, in the endoscopic image 40.

The plurality of segmentation images 44 displayed in the second display region 38 are images corresponding to the endoscopic image 40 and are referred to by the doctor 16 to identify the positions of the lesions 42 in the endoscopic image 40.

While the plurality of segmentation images 44 are illustrated here, a plurality of bounding boxes are displayed instead of the plurality of segmentation images 44 in a case where the lesions 42 are recognized by performing an object recognition process using an AI-based bounding box method on the endoscopic image 40. Alternatively, the plurality of segmentation images 44 and the plurality of bounding boxes may be used in combination. The segmentation images 44 and the bounding boxes are merely examples, and any images may be used so long as the positional relationship of the plurality of lesions 42 appearing in the endoscopic image 40 can be identified.

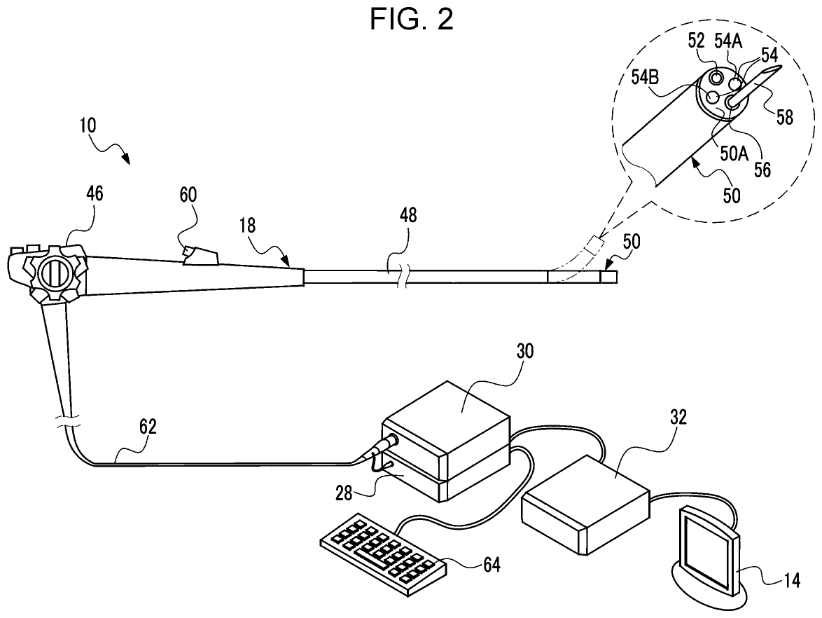

As an example, as illustrated in FIG. 2, the endoscope main body 18 includes an operation section 46 and an insertion section 48. The insertion section 48 partially bends in response to the operation section 46 being operated. When the doctor 16 (see FIG. 1) operates the operation section 46, the insertion section 48 is inserted into the large intestine 22 (see FIG. 1) while bending according to the shape of the large intestine 22.

The insertion section 48 has a tip portion 50 provided with a camera 52, an illumination device 54, and a treatment tool opening 56. The camera 52 and the illumination device 54 are provided on a tip surface 50A of the tip portion 50. While an example embodiment in which the camera 52 and the illumination device 54 are provided on the tip surface 50A of the tip portion 50 is given here, this is merely an example. The camera 52 and the illumination device 54 may be provided on a side surface of the tip portion 50 such that the endoscope 12 is configured as a side view endoscope.

The camera 52 is a device that performs imaging of the inside of the body (for example, the inside of the large intestine 22) of the subject 20 to acquire the endoscopic image 40 as a medical image. An example of the camera 52 is a CMOS camera. However, this is merely an example, and the camera 52 may be any other type of camera such as a CCD camera. The camera 52 is an example of a “module” according to the technology of the present disclosure.

The illumination device 54 has illumination windows 54A and 54B. The illumination device 54 emits the light 26 (see FIG. 1) through the illumination windows 54A and 54B. Types of the light 26 to be emitted from the illumination device 54 include, for example, visible light (for example, white light or the like) and invisible light (for example, near-infrared light or the like). The illumination device 54 further emits special light through the illumination windows 54A and 54B. Examples of the special light include light for BLI and/or light for LCI. The camera 52 performs imaging of the inside of the large intestine 22 using an optical method, with the inside of the large intestine 22 irradiated with the light 26 from the illumination device 54.

The treatment tool opening 56 is an opening for allowing a treatment tool 58 to protrude from the tip portion 50. The treatment tool opening 56 is also used as a suction port for sucking blood, bodily waste, and the like, and as a delivery port for delivering a fluid.

The operation section 46 has a treatment tool insertion port 60 formed therein, and the treatment tool 58 is inserted into the insertion section 48 through the treatment tool insertion port 60. The treatment tool 58 passes through the insertion section 48 and protrudes to the outside from the treatment tool opening 56. In the example illustrated in FIG. 2, a puncture needle is illustrated as the treatment tool 58 protruding from the treatment tool opening 56. While a puncture needle is illustrated as the treatment tool 58, this is merely an example, and the treatment tool 58 may be gripping forceps, a papillotomy knife, a snare, a catheter, a guide wire, a cannula, a puncture needle with a guide sheath, and/or the like.

The endoscope main body 18 is connected to the control device 28 and the light source device 30 through a universal cord 62. The control device 28 is connected to the image processing device 32 and a reception device 64. The image processing device 32 is further connected to the display device 14. That is, the control device 28 is connected to the display device 14 through the image processing device 32.

Since the image processing device 32 is illustrated here as an external device for extending the functions implemented by the control device 28, an example embodiment in which the control device 28 and the display device 14 are indirectly connected through the image processing device 32 is given here, although this is merely an example. For example, the display device 14 may be directly connected to the control device 28. In this case, for example, the control device 28 may be equipped with the functions of the image processing device 32, or the control device 28 may be equipped with a function of causing a server (not illustrated) to execute the same process as a process executed by the image processing device 32 (for example, a medical support process described below) and receiving and using a processing result obtained by the server.

The reception device 64 receives an instruction from the doctor 16 and outputs the received instruction to the control device 28 as an electrical signal. An example of the reception device 64 is a keyboard, a mouse, a touch panel, a foot switch, a microphone, and/or a remote operation device.

The control device 28 controls the light source device 30, transmits and receives various signals to and from the camera 52, and transmits and receives various signals to and from the image processing device 32.

The light source device 30 emits light under the control of the control device 28 and supplies the light to the illumination device 54. The illumination device 54 incorporates a light guide, and the light supplied from the light source device 30 is emitted from the illumination windows 54A and 54B through the light guide. The control device 28 causes the camera 52 to perform imaging, acquires the endoscopic image 40 (see FIG. 1) from the camera 52, and outputs the endoscopic image 40 to a specified output destination (for example, the image processing device 32).

The image processing device 32 performs various kinds of image processing on the endoscopic image 40 input from the control device 28. The image processing device 32 outputs the endoscopic image 40 on which the various kinds of image processing have been performed to a specified output destination (for example, the display device 14).

While an example embodiment has been described in which the endoscopic image 40 output from the control device 28 is output to the display device 14 through the image processing device 32, this is merely an example. For example, in another aspect, the control device 28 and the display device 14 may be connected to each other, and the endoscopic image 40 on which image processing has been performed by the image processing device 32 may be displayed on the display device 14 through the control device 28.

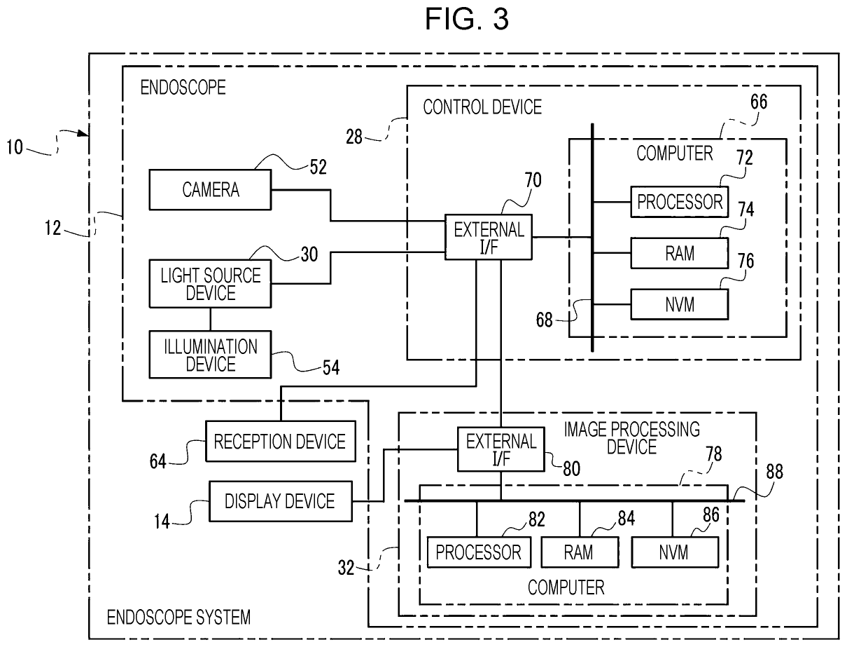

As an example, as illustrated in FIG. 3, the control device 28 includes a computer 66, a bus 68, and an external I/F 70. The computer 66 includes a processor 72, a RAM 74, and an NVM 76. The processor 72, the RAM 74, the NVM 76, and the external I/F 70 are connected to the bus 68.

For example, the processor 72 has at least one CPU and at least one GPU and controls the entire control device 28. The GPU operates under the control of the CPU and is responsible for performing various kinds of graphics-based processing, arithmetic operations using a neural network, and the like. The processor 72 may include one or more CPUs with integrated GPU functions, or may include one or more CPUs without integrated GPU functions. In the example illustrated in FIG. 3, the computer 66 is mounted with one processor 72. However, this is merely an example, and the computer 66 may be mounted with a plurality of processors 72.

The RAM 74 is a memory that temporarily stores information and is used as a work memory by the processor 72. The NVM 76 is a non-volatile storage device that stores various programs, various parameters, and the like. An example of the NVM 76 is a flash memory (for example, an EEPROM and/or an SSD). The flash memory is merely an example, and the NVM 76 may be any other non-volatile storage device such as an HDD, or a combination of two or more types of non-volatile storage devices.

The external I/F 70 handles transmission and reception of various kinds of information between the processor 72 and one or more devices (hereinafter also referred to as “first external devices”) external to the control device 28. An example of the external I/F 70 is a USB interface.

The camera 52 is connected to the external I/F 70 as one of the first external devices, and the external I/F 70 handles transmission and reception of various kinds of information between the camera 52 and the processor 72. The processor 72 controls the camera 52 through the external I/F 70. Further, the processor 72 acquires the endoscopic image 40 (see FIG. 1), which is obtained by imaging of the inside of the large intestine 22 (see FIG. 1) using the camera 52, through the external I/F 70.

The light source device 30 is connected to the external I/F 70 as one of the first external devices, and the external I/F 70 handles transmission and reception of various kinds of information between the light source device 30 and the processor 72. The light source device 30 supplies light to the illumination device 54 under the control of the processor 72. The illumination device 54 emits the light supplied from the light source device 30.

The reception device 64 is connected to the external I/F 70 as one of the first external devices, and the processor 72 acquires an instruction received by the reception device 64 through the external I/F 70 and executes a process corresponding to the acquired instruction.

The image processing device 32 includes a computer 78 and an external I/F 80. The computer 78 includes a processor 82, a RAM 84, and an NVM 86. The processor 82, the RAM 84, the NVM 86, and the external I/F 80 are connected to a bus 88. In the present embodiment, the image processing device 32 is an example of a “medical support device” according to the technology of the present disclosure, the computer 78 is an example of a “computer” according to the technology of the present disclosure, and the processor 82 is an example of a “processor” according to the technology of the present disclosure.

Since the hardware configuration (that is, the processor 82, the RAM 84, and the NVM 86) of the computer 78 is basically the same as the hardware configuration of the computer 66, the description of the hardware configuration of the computer 78 will be omitted here.

The external I/F 80 handles transmission and reception of various kinds of information between the processor 82 and one or more devices (hereinafter also referred to as “second external devices”) external to the image processing device 32. An example of the external I/F 80 is a USB interface.

The control device 28 is connected to the external I/F 80 as one of the second external devices. In the example illustrated in FIG. 3, the external I/F 70 of the control device 28 is connected to the external I/F 80. The external I/F 80 handles transmission and reception of various kinds of information between the processor 82 of the image processing device 32 and the processor 72 of the control device 28. For example, the processor 82 acquires the endoscopic image 40 (see FIG. 1) from the processor 72 of the control device 28 through the external I/Fs 70 and 80, and performs various kinds of image processing on the acquired endoscopic image 40.

The display device 14 is connected to the external I/F 80 as one of the second external devices. The processor 82 controls the display device 14 through the external I/F 80 to display various kinds of information (for example, the endoscopic image 40 and the like on which the various kinds of image processing have been performed) on the display device 14.

In an endoscopic examination, the doctor 16 determines whether the plurality of lesions 42 appearing in the endoscopic image 40 require medical treatment, while checking the endoscopic image 40 through the display device 14, and performs medical treatment on the plurality of lesions 42, if necessary. The sizes of the plurality of lesions 42 are a determination factor important for determining whether medical treatment is necessary.

The recent development of machine learning has enabled the AI-based detection and classification of the plurality of lesions 42 based on the endoscopic image 40. Application of this technique makes it possible to measure the sizes of the plurality of lesions 42 from the endoscopic image 40.

However, when the plurality of lesions 42 appear in the endoscopic image 40, which of the lesions 42 the doctor 16 is interested in differs depending on the positions of the lesions 42 in the endoscopic image 40. Presenting the size of the lesion 42 of low interest to the doctor 16 preferentially to the doctor 16 or presenting the size of the lesion 42 of high interest to the doctor 16 and the size of the lesion 42 of low interest to the doctor 16 indistinguishably to the doctor 16 may be inefficient for performing an endoscopic examination, or may cause confusion. By contrast, it can be said that preferentially measuring the size of the lesion 42 of high interest to the doctor 16 and presenting the measured size to the doctor 16 or presenting the size of the lesion 42 of high interest to the doctor 16 and the size of the lesion 42 of low interest to the doctor 16 distinguishably to the doctor 16 are useful for performing an endoscopic examination.

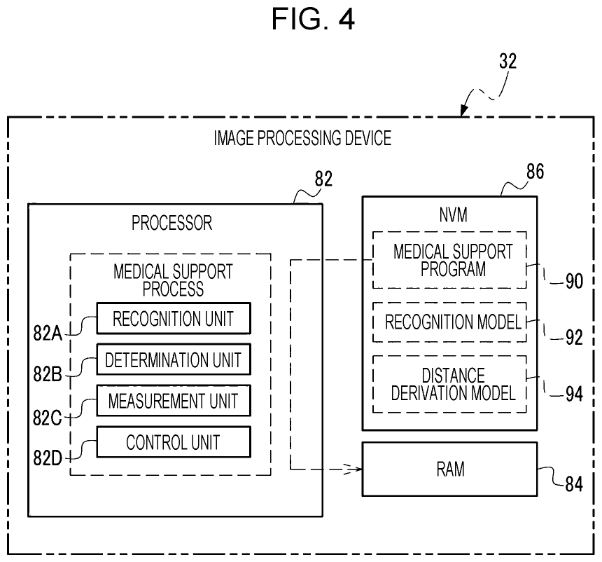

In view of such circumstances, in the present embodiment, as an example, as illustrated in FIG. 4, the processor 82 of the image processing device 32 performs a medical support process.

The NVM 86 stores a medical support program 90. The medical support program 90 is an example of a “program” according to the technology of the present disclosure. The processor 82 reads the medical support program 90 from the NVM 86 and executes the read medical support program 90 on the RAM 84 to perform the medical support process. The medical support process is implemented by the processor 82 operating as a recognition unit 82A, a determination unit 82B, a measurement unit 82C, and a control unit 82D in accordance with the medical support program 90 executed on the RAM 84.

The NVM 86 stores a recognition model 92 and a distance derivation model 94. The recognition model 92 and the distance derivation model 94 are examples of an “AI” according to the technology of the present disclosure. As described in detail below, the recognition model 92 is used by the recognition unit 82A, and the distance derivation model 94 is used by the measurement unit 82C.

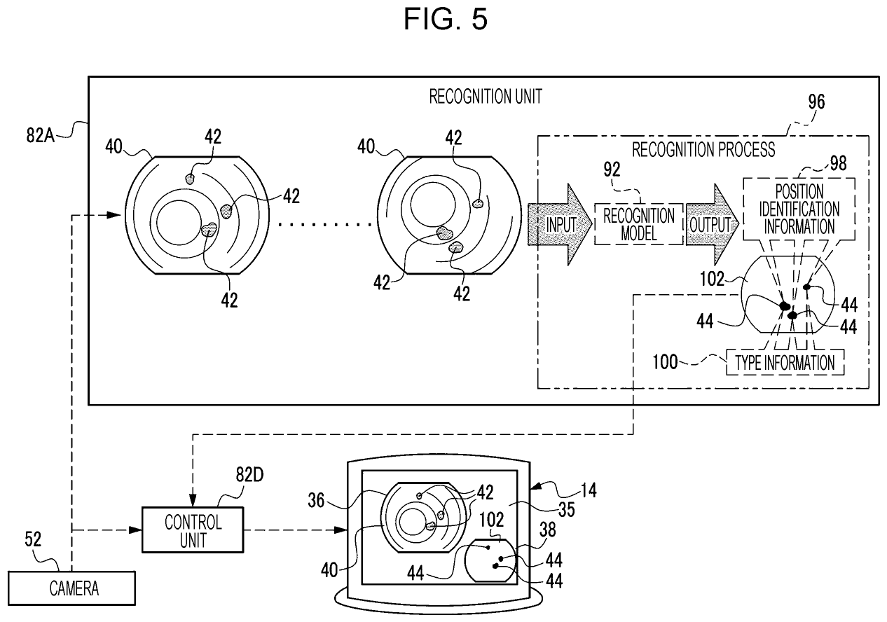

As an example, as illustrated in FIG. 5, the recognition unit 82A and the control unit 82D acquire an endoscopic image 40, which is generated through imaging performed by the camera 52 in accordance with an imaging frame rate (for example, several tens of frames/second), from the camera 52 on a frame-by-frame basis.

The control unit 82D displays the endoscopic image 40 in the first display region 36 as a live view image. That is, each time the control unit 82D acquires an endoscopic image 40 from the camera 52 on a frame-by-frame basis, the control unit 82D sequentially displays the acquired endoscopic image 40 in the first display region 36 in accordance with a display frame rate (for example, several tens of frames/second).

The recognition unit 82A performs a recognition process 96 on the endoscopic image 40 acquired from the camera 52 to recognize the positions of the lesions 42 in the endoscopic image 40 (that is, the positions of the lesions 42 appearing in the endoscopic image 40) and the types of the lesions 42. Each time the recognition unit 82A acquires the endoscopic image 40, the recognition process 96 is performed on the acquired endoscopic image 40.

The recognition process 96 is an object recognition process using an AI-based segmentation method. A process using the recognition model 92 is performed as the recognition process 96.

The recognition model 92 is a trained model for object recognition using an AI-based segmentation method, and is optimized by training a neural network through machine learning using first training data. The first training data is a dataset including a plurality of pieces of data (that is, data for a plurality of frames) in which first example data and first ground-truth data are associated with each other.

The first example data is an image corresponding to the endoscopic image 40. The first ground-truth data is ground-truth data (that is, an annotation) for the first example data. An example of the first ground-truth data is an annotation for identifying the position and type of a lesion appearing in an image used as the first example data.

The recognition unit 82A acquires the endoscopic image 40 from the camera 52 and inputs the acquired endoscopic image 40 to the recognition model 92. Accordingly, each time the endoscopic image 40 is input, the recognition model 92 identifies, as the positions of the lesions 42 appearing in the input endoscopic image 40, the positions of the segmentation images 44 identified by the segmentation method and outputs position identification information 98 that can identify the positions of the segmentation images 44. Examples of the position identification information 98 include coordinates for identifying the segmentation images 44 in the endoscopic image 40. Each time the endoscopic image 40 is input, the recognition model 92 further recognizes the types of the lesions 42 (for example, names of lesions (as an example, NP, SSL, HP, and the like)) appearing in the input endoscopic image 40 and outputs type information 100 indicating the recognized types. The segmentation images 44 are associated with the position identification information 98 and the type information 100.

The control unit 82D displays, in the second display region 38, a map 102 indicating the distribution of the positions of the plurality of lesions 42 for each endoscopic image 40 in accordance with the position identification information 98 and the plurality of segmentation images 44. The map 102 is created by the recognition unit 82A. The distribution of the positions of the plurality of lesions 42 for each endoscopic image 40 on the map 102 is represented by the plurality of segmentation images 44 obtained for each endoscopic image 40 by the recognition unit 82A. For example, the map 102 displayed in the second display region 38 is updated in accordance with the display frame rate applied to the first display region 36. That is, the display of the plurality of segmentation images 44 in the second display region 38 is updated in synchronization with the display timing of the endoscopic image 40 displayed in the first display region 36. This allows the doctor 16 to grasp the schematic positions of the plurality of lesions 42 in the endoscopic image 40 displayed in the first display region 36 by referring to the map 102 displayed in the second display region 38 while observing the endoscopic image 40 displayed in the first display region 36. In the present embodiment, the map 102 is an example of a “map” according to the technology of the present disclosure.

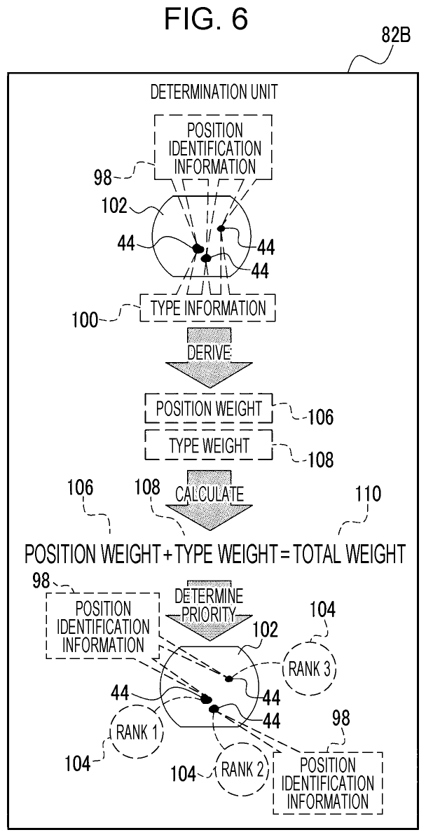

As an example, as illustrated in FIG. 6, each time the recognition unit 82A performs the recognition process 96 (see FIG. 5) on each endoscopic image 40, the determination unit 82B acquires the position identification information 98, the type information 100, and the map 102 including the plurality of segmentation images 44 from the recognition unit 82A. Then, the determination unit 82B determines a priority 104 of the plurality of lesions 42, based on the position identification information 98 acquired from the recognition unit 82A. The priority 104 is the order of priorities (in other words, importance) assigned to the plurality of lesions 42 and is determined such that a high priority is assigned to the lesion 42 expected to be of high interest to the doctor 16 and a low priority is assigned to the lesion 42 expected to be of low interest to the doctor 16.

In the example illustrated in FIG. 6, the priority 104 is determined by the determination unit 82B, based on the position identification information 98 and the type information 100.

To this end, first, the determination unit 82B derives a position weight 106 based on the position identification information 98, and derives a type weight 108 based on the type information 100.

The position weight 106 is a weight (that is, a priority level (in other words, a level of importance)) for each of the positions of the lesions 42 in the endoscopic image 40. For example, the position weight 106 increases as the distance to the center of the endoscopic image 40 decreases. For example, when the value of the position weight 106 is represented by “x”, the position weight 106 is a value determined within the range of “0≤x≤0.5”. A first example of a means for deriving the position weight 106 from the position identification information 98 is a means that uses an arithmetic expression in which the position identification information 98 is a dependent variable and the position weight 106 is an independent variable. A second example of the means for deriving the position weight 106 from the position identification information 98 is a means that uses a table with the position identification information 98 as an input and the position weight 106 as an output.

The type weight 108 is the type (that is, a priority level (in other words, a level of importance)) of the lesion 42 indicated by the type information 100. For example, the type weight 108 increases as the severity of the type of the lesion 42 increases. For example, the type weight 108 for neoplastic polyp is larger than the type weight 108 for non-neoplastic polyp. For example, when the value of the type weight 108 is represented by “y”, the type weight 108 is a value determined within the range of “0≤y≤0.5”. A first example of a means for deriving the type weight 108 from the type information 100 is a means that uses a table with the type information 100 as an input and the type weight 108 as an output. A second example of the means for deriving the type weight 108 from the type information 100 is a means that uses an arithmetic expression in which the type information 100 is a dependent variable and the type weight 108 is an independent variable on the premise that the type information 100 is represented by a variable that can identify the type of the lesion 42.

The determination unit 82B calculates a total weight 110 based on the position weight 106 and the type weight 108. In the example illustrated in FIG. 6, the sum of the position weight 106 and the type weight 108 is presented as the total weight 110.

The sum of the position weight 106 and the type weight 108 is merely an example, and the total weight 110 may be the product of the position weight 106 and the type weight 108. At least one of the position weight 106 or the type weight 108 may be multiplied by a coefficient. The coefficient may be a fixed value, or may be a variable value.

When the coefficient is a variable value, the coefficient may be determined in accordance with various conditions (for example, the type of the endoscopic examination, the specifications of the endoscope 12, the user of the endoscope 12, and/or the like), or may be determined in accordance with an instruction given from the doctor 16 or the like through the reception device 64. The position weight 106 itself and/or the type weight 108 itself may be changed in a similar manner.

The determination unit 82B determines the priority 104 of the plurality of lesions 42, based on the total weight 110 calculated for each of the plurality of lesions 42. The priority 104 increases as the total weight 110 increases. For example, the sum of the position weight 106 and the type weight 108 is used as the total weight 110. When a focus is placed on the position weight 106, the total weight 110 increases as the position weight 106 increases, and, accordingly, the priority 104 increases. In the present embodiment, the position weight 106 increases as the distance to the center of the endoscopic image 40 decreases. For this reason, the priority 104 is higher for the positions of lesions 42 closer to the center of the endoscopic image 40. When a focus is placed on the type weight 108, the total weight 110 increases as the type weight 108 increases, and, accordingly, the priority 104 increases. In the present embodiment, the type weight 108 increases as the severity of the lesion 42 increases. For this reason, the priority 104 increases as the severity of the lesion 42 increases.

The determination unit 82B assigns the determined priority 104 to the plurality of lesions 42. In the example illustrated in FIG. 6, the assignment of the priority 104 to the plurality of lesions 42 is implemented by assigning the priority 104 determined by the determination unit 82B to each of the plurality of segmentation images 44 corresponding to the plurality of lesions 42.

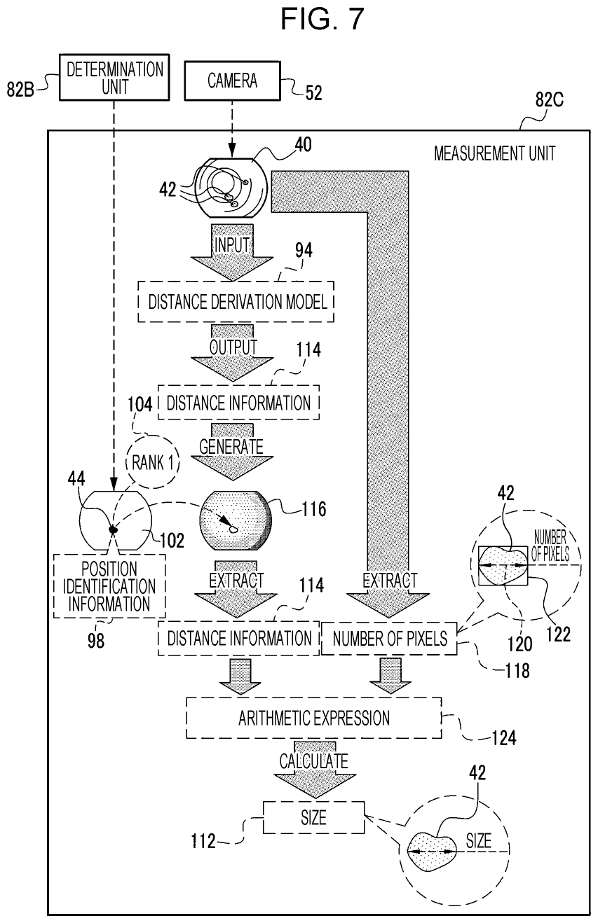

When the priority 104 of the plurality of lesions 42 is determined by the determination unit 82B, as an example, as illustrated in FIG. 7, the measurement unit 82C measures sizes 112 of the lesions 42, based on the endoscopic image 40 acquired from the camera 52 (for example, the endoscopic image 40 used by the recognition unit 82A to obtain the plurality of segmentation images 44, the position identification information 98, and the type information 100 used by the determination unit 82B). The measurement unit 82C measures the sizes 112 of the plurality of lesions 42 in accordance with the priority 104 determined by the determination unit 82B. That is, the sizes 112 of the lesions 42 are measured in order from the lesion 42 having the highest priority 104 to the lesion 42 having the lowest priority 104.

The measurement unit 82C acquires distance information 114 of the plurality of lesions 42, based on the endoscopic image 40 acquired from the camera 52. The distance information 114 is information indicating the distance from the camera 52 (that is, the observation position) to the intestinal wall 24 (see FIG. 1) including the lesions 42. The distance from the camera 52 to the intestinal wall 24 including the lesions 42 is an example of a “depth” according to the technology of the present disclosure. While the distance from the camera 52 to the intestinal wall 24 including the lesions 42 is illustrated here, this is merely an example. Instead of the distance, a numerical value indicating the depth from the camera 52 to the intestinal wall 24 including the lesions 42 (for example, a plurality of numerical values defining the depth in steps (for example, numerical values in several steps to several tens of steps)) may be used.

The distance information 114 is acquired for each of all the pixels constituting the endoscopic image 40. The distance information 114 may be acquired for each block (for example, a pixel group constituted by several to several hundreds of pixels), which is larger than a pixel in the endoscopic image 40.

The measurement unit 82C acquires the distance information 114 by, for example, deriving the distance information 114 by using an AI-based method. In the present embodiment, the distance derivation model 94 is used to derive the distance information 114.

The distance derivation model 94 is optimized by training the neural network through machine learning using second training data.

The second training data is a dataset including a plurality of pieces of data (that is, data for a plurality of frames) in which second example data and second ground-truth data are associated with each other.

The second example data is an image corresponding to the endoscopic image 40. The second ground-truth data is ground-truth data (that is, an annotation) for the second example data. An example of the second ground-truth data is an annotation for identifying a distance corresponding to each pixel appearing in an image used as the second example data.

The measurement unit 82C acquires the endoscopic image 40 from the camera 52 and inputs the acquired endoscopic image 40 to the distance derivation model 94. As a result, the distance derivation model 94 outputs the distance information 114 on a pixel-by-pixel basis in the input endoscopic image 40. That is, in the measurement unit 82C, information indicating the distance from the position of the camera 52 (for example, the position of the image sensor, the objective lens, or the like mounted in the camera 52) to the intestinal wall 24 appearing in the endoscopic image 40 is output from the distance derivation model 94 as the distance information 114 on a pixel-by-pixel basis in the endoscopic image 40.

The measurement unit 82C generates a distance image 116, based on the distance information 114 output from the distance derivation model 94. The distance image 116 is an image in which the distance information 114 is distributed in units of pixels included in the endoscopic image 40.

The measurement unit 82C acquires the position identification information 98 from the determination unit 82B in accordance with the priority 104 determined by the determination unit 82B. That is, the measurement unit 82C acquires the position identification information 98 assigned to the segmentation images 44 in a sequence based on the priority 104, from highest to lowest. For example, in the example illustrated in FIG. 6, three segmentation images 44 are assigned ranks 1 through 3 based on the priority 104. Thus, the measurement unit 82C sequentially acquires the position identification information 98, starting from the position identification information 98 assigned to the segmentation image 44 ranked first in the priority 104 to the position identification information 98 assigned to the segmentation image 44 ranked third in the priority 104.

The measurement unit 82C sequentially acquires the position identification information 98 from the plurality of segmentation images 44 in a sequence based on the priority 104, from highest to lowest, refers to the acquired position identification information 98, and extracts, from the distance image 116, the distance information 114 corresponding to the position identified from the position identification information 98. Examples of the distance information 114 extracted from the distance image 116 include the distance information 114 corresponding to a specific position (for example, centroid) of the lesion 42, and the statistical value (for example, median value, mean value, or mode value) of the distance information 114 for a plurality of pixels (for example, all the pixels) included in the lesion 42.

The measurement unit 82C extracts the number of pixels 118 from the endoscopic image 40. The number of pixels 118 is the number of pixels on a line segment 120 crossing an image region at a position identified from the position identification information 98 (that is, an image region indicating the lesion 42) within an entire image region of the endoscopic image 40 input to the distance derivation model 94. An example of the line segment 120 is the longest line segment parallel to the long sides of a circumscribed rectangular frame 122 of the image region indicating the lesion 42. The line segment 120 is merely an example. Instead of the line segment 120, the longest line segment parallel to the short sides of the circumscribed rectangular frame 122 of the image region indicating the lesion 42 may be used.

The measurement unit 82C calculates the size 112 of the lesion 42 in real space, based on the distance information 114 extracted from the distance image 116 and the number of pixels 118 extracted from the endoscopic image 40. The size 112 refers to, for example, the length of the lesion 42 in real space.

The size 112 is calculated using an arithmetic expression 124. The measurement unit 82C inputs the distance information 114 extracted from the distance image 116 and the number of pixels 118 extracted from the endoscopic image 40 to the arithmetic expression 124. The arithmetic expression 124 is an arithmetic expression in which the distance information 114 and the number of pixels 118 are independent variables and the size 112 is a dependent variable. The arithmetic expression 124 outputs the size 112 corresponding to the input distance information 114 and the input number of pixels 118.

The example illustrated in FIG. 7 provides an example embodiment in which the size 112 of the lesion 42 corresponding to the segmentation image 44 assigned rank 1 based on the priority 104 is measured by the measurement unit 82C. However, the sizes 112 of the lesions 42 corresponding to the segmentation images 44 assigned ranks 2 and 3 based on the priority 104 illustrated in FIG. 6 are also measured by the measurement unit 82C in a sequence based on the priority 104, from highest to lowest.

While an example embodiment has been described in which the sizes 112 of the plurality of lesions 42 are sequentially measured in an order based on the priority 104, the technology of the present disclosure is not limited to this. The sizes 112 of the plurality of lesions 42 may be measured in parallel.

While the length of each lesion 42 in real space is illustrated as the size 112, the technology of the present disclosure is not limited to this. The size 112 may be the surface area or volume of each lesion 42 in real space. In this case, for example, as the arithmetic expression 124, an arithmetic expression is used in which the number of pixels in an entire image region indicating each lesion 42 and the distance information 114 are independent variables and the surface area or volume of each lesion 42 in real space is a dependent variable.

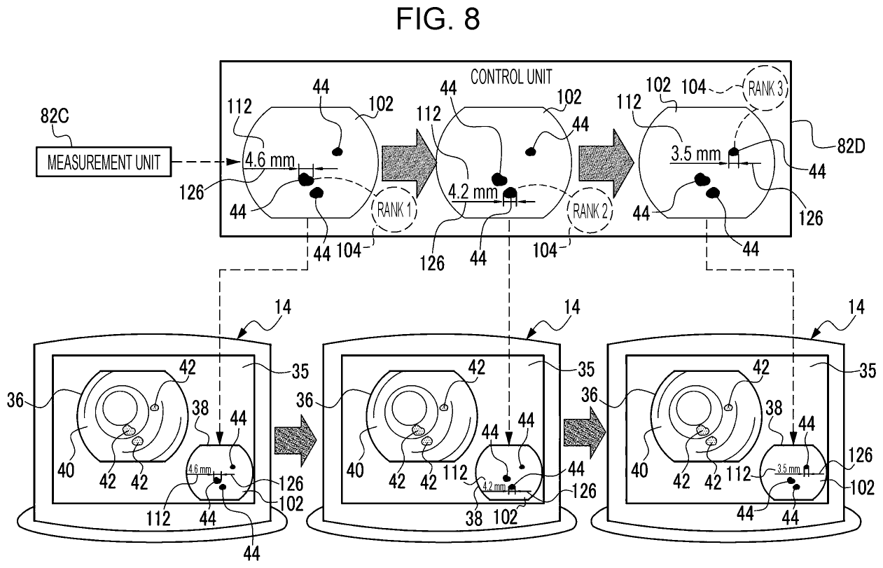

As an example, as illustrated in FIG. 8, the control unit 82D displays the map 102 in the second display region 38. Then, the control unit 82D displays the sizes 112 on the map 102 in accordance with the priority 104 assigned to the plurality of segmentation images 44. For example, the sizes 112 are superimposed and displayed on the map 102. The superimposed display is merely an example, and embedded display may be used.

The map 102 displays the plurality of segmentation images 44, and the sizes 112 measured by the measurement unit 82C are displayed in a sequence based on the priority 104 from highest to lowest (in the example illustrated in FIG. 8, from rank 1 to rank 3). That is, the size 112 displayed on the map 102 is switched in accordance with the priority 104. The time interval at which the size 112 is switched, that is, the time period during which each size 112 is continuously displayed in accordance with the priority 104, may be a fixed time period of about several seconds to several tens of seconds or may be a variable time period that can be changed in accordance with an instruction given from the doctor 16 or the like through the reception device 64.

The control unit 82D displays a dimension line 126 on the map 102 as information that can identify which lesion 42 among the plurality of lesions 42 the size 112 to be displayed on the map 102 corresponds to. The dimension line 126 is a mark that enables the identification of a portion of the segmentation image 44 to which the size 112 corresponds. The dimension line 126 is generated and displayed by, for example, the control unit 82D, based on the position identification information 98 acquired from the recognition unit 82A. It is sufficient that, for example, the dimension line 126 be generated in a manner similar to that for the generation of the line segment 120 (that is, in a manner similar to that using the circumscribed rectangular frame 122). In the present embodiment, the dimension line 126 is an example of “region identification information” according to the technology of the present disclosure.

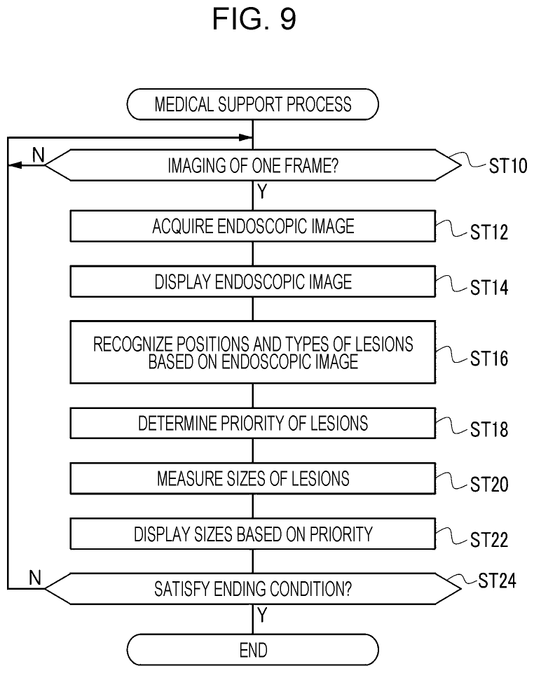

Next, the operation of a portion, according to the technology of the present disclosure, of the endoscope system 10 will be described with reference to FIG. 9. The flow of the medical support process illustrated in FIG. 9 is an example of a “medical support method” according to the technology of the present disclosure.

In the medical support process illustrated in FIG. 9, first, in step ST10, the recognition unit 82A determines whether imaging of one frame has been performed in the large intestine 22 by the camera 52. If imaging of one frame has not been performed in the large intestine 22 by the camera 52 in step ST10, the determination is negative, and the determination of step ST10 is performed again. If imaging of one frame has been performed in the large intestine 22 by the camera 52 in step ST10, the determination is affirmative, and the medical support process proceeds to step ST12.

In step ST12, the recognition unit 82A and the control unit 82D acquire an endoscopic image 40 corresponding to one frame, which is obtained by imaging of the large intestine 22 using the camera 52 (see FIG. 5). For convenience of description, it is assumed here that a plurality of lesions 42 appear in the endoscopic image 40. After the processing of step ST12 is performed, the medical support process proceeds to step ST14.

In step ST14, the control unit 82D displays the endoscopic image 40 acquired in step ST12 in the first display region 36 (see FIGS. 1, 5, and 8). After the processing of step ST14 is performed, the medical support process proceeds to step ST16.

In step ST16, the recognition unit 82A performs the recognition process 96 using the endoscopic image 40 acquired in step ST12 to recognize the positions and types of the plurality of lesions 42 in the endoscopic image 40, and acquires the position identification information 98 and the type information 100 (see FIG. 5). After the processing of step ST16 is performed, the medical support process proceeds to step ST18.

In step ST18, the determination unit 82B determines the priority 104 of the plurality of lesions 42 appearing in the endoscopic image 40 acquired in step ST12, based on the position identification information 98 and the type information 100 acquired by the recognition unit 82A in step ST16 (see FIG. 6). After the processing of step ST18 is performed, the medical support process proceeds to step ST20.

In step ST20, the measurement unit 82C measures the sizes 112 of the plurality of lesions 42 appearing in the endoscopic image 40 acquired in step ST12 (see FIG. 7). After the processing of step ST20 is performed, the medical support process proceeds to step ST22.

In step ST22, the control unit 82D displays the plurality of sizes 112 of the plurality of lesions 42 measured by the measurement unit 82C in step ST20 in the second display region 38 in accordance with the priority 104 determined in step ST18 (see FIG. 8). After the processing of step ST22 is performed, the medical support process proceeds to step ST24.

In step ST24, the control unit 82D determines whether a condition for ending the medical support process is satisfied. An example of the condition for ending the medical support process is a condition in which an instruction to end the medical support process is given to the endoscope system 10 (for example, a condition in which the instruction to end the medical support process is received by the reception device 64).

If the condition for ending the medical support process is not satisfied in step ST24, the determination is negative, and the medical support process returns to step ST10. If the condition for ending the medical support process is satisfied in step ST24, the determination is affirmative, and the medical support process ends.

As described above, in the endoscope system 10 according to the present embodiment, the recognition unit 82A recognizes the positions of the plurality of lesions 42 in the endoscopic image 40, based on the endoscopic image 40 in which the plurality of lesions 42 appear. Further, the determination unit 82B determines the priority 104 of the plurality of lesions 42, based on the positions of the plurality of lesions 42 in the endoscopic image 40. Further, the measurement unit 82C measures the sizes 112 of the plurality of lesions 42. Then, the control unit 82D displays the sizes 112 on the screen 35 in accordance with the priority 104. In the present embodiment, as an example, the priority 104 is higher for lesions 42 whose positions are closer to the center of the endoscopic image 40. Accordingly, in a case where the lesion 42 whose position in the endoscopic image 40 is closer to the center of the endoscopic image 40 is of higher interest to the doctor 16, the doctor 16 can grasp the size 112 of the lesion 42 expected to be of high interest to the doctor 16 among the plurality of lesions 42 appearing in the endoscopic image 40.

In the endoscope system 10 according to the present embodiment, furthermore, the sizes 112 of the lesions 42 are displayed on the screen 35 one at a time in an order based on the priority 104. This allows the doctor 16 to grasp the sizes 112 of the lesions 42 sequentially, starting from the size 112 of the lesion 42 expected to be of highest interest to the doctor 16 to the size 112 of the lesion 42 expected to be of lowest interest to the doctor 16 among the plurality of lesions 42 appearing in the endoscopic image 40.

In the endoscope system 10 according to the present embodiment, furthermore, a size 112 is displayed on the screen 35. Accordingly, in a case where a plurality of lesions 42 appear in the endoscopic image 40, the doctor 16 can visually recognize the size 112 of the lesion 42 expected to be of high interest to the doctor 16.

In the endoscope system 10 according to the present embodiment, furthermore, the endoscopic image 40 is displayed in the first display region 36 on the screen 35. Further, the map 102 is displayed in the second display region 38 on the screen 35, and the dimension line 126 is displayed on the map 102 as information that can identify the lesion 42 corresponding to the size 112 displayed in the second display region 38. This allows the doctor 16 to visually recognize, through the map 102, which lesion 42 the size 112 displayed on the screen 35 corresponds to among the plurality of lesions 42 appearing in the endoscopic image 40.

In the endoscope system 10 according to the present embodiment, furthermore, the size 112 to be displayed on the screen 35 is switched in accordance with the priority 104. Accordingly, in a case where a plurality of lesions 42 appear in the endoscopic image 40, the doctor 16 can visually recognize the sizes 112 of the lesions 42 sequentially, starting from the size 112 of the lesion 42 expected to be of highest interest to the doctor 16 to the size 112 of the lesion 42 expected to be of lowest interest to the doctor 16.

In the endoscope system 10 according to the present embodiment, furthermore, the priority 104 is higher for the positions of lesions 42 closer to the center of the endoscopic image 40. This allows the doctor 16 to grasp the size 112 of the lesion 42 of high interest to the doctor 16 when the doctor 16 is interested in the center portion of the endoscopic image 40.

In the endoscope system 10 according to the present embodiment, furthermore, the recognition unit 82A recognizes the positions and types of the plurality of lesions 42 in the endoscopic image 40, based on the endoscopic image 40 in which the plurality of lesions 42 appear. Further, the determination unit 82B determines the priority 104 of the plurality of lesions 42, based on the positions and types of the plurality of lesions 42 in the endoscopic image 40. Then, the control unit 82D displays the sizes 112 on the screen 35 in accordance with the priority 104. In the present embodiment, as an example, the priority 104 is higher for lesions 42 whose positions are closer to the center of the endoscopic image 40, and is higher for the lesions 42 whose types have higher severity. Accordingly, in a case where a plurality of lesions 42 appearing in the endoscopic image 40 include both a lesion 42 whose type is of high interest to the doctor 16 and a lesion 42 whose type is of low interest to the doctor 16, the doctor 16 can grasp the size 112 of the lesion 42 whose type is of high interest to the doctor 16 and the size 112 of the lesion 42 whose type is not of high interest to the doctor 16.

In the endoscope system 10 according to the present embodiment, furthermore, the measurement unit 82C measures the sizes 112 of the plurality of lesions 42 in accordance with the priority 104. Accordingly, the size 112 of the lesion 42 of high interest to the doctor 16 among the plurality of lesions 42 appearing in the endoscopic image 40 can be preferentially measured. This can result in the size 112 of the lesion 42 of high interest to the doctor 16 being quickly presented to the doctor 16.

While the embodiment described above describes a case where the position weight 106 increases as the distance to the center of the endoscopic image 40 decreases, the technology of the present disclosure is not limited to this. For example, the position weight 106 may be set to increase as the distance to a designated position in the endoscopic image 40 (for example, a region designated by the doctor 16 as a region to be gazed at by the doctor 16) decreases. Alternatively, the position weight 106 may be set to decrease as the distance to a designated position in the endoscopic image 40 (for example, a region determined as a region affected by the optical effect (for example, distortion) of the lens of the camera 52 in the endoscopic image 40 (for example, a marginal portion of the endoscopic image 40)) decreases. The designated position in the endoscopic image 40 may be a fixed position determined in advance in accordance with various conditions, or may be a position changed in accordance with various conditions and/or a given instruction.

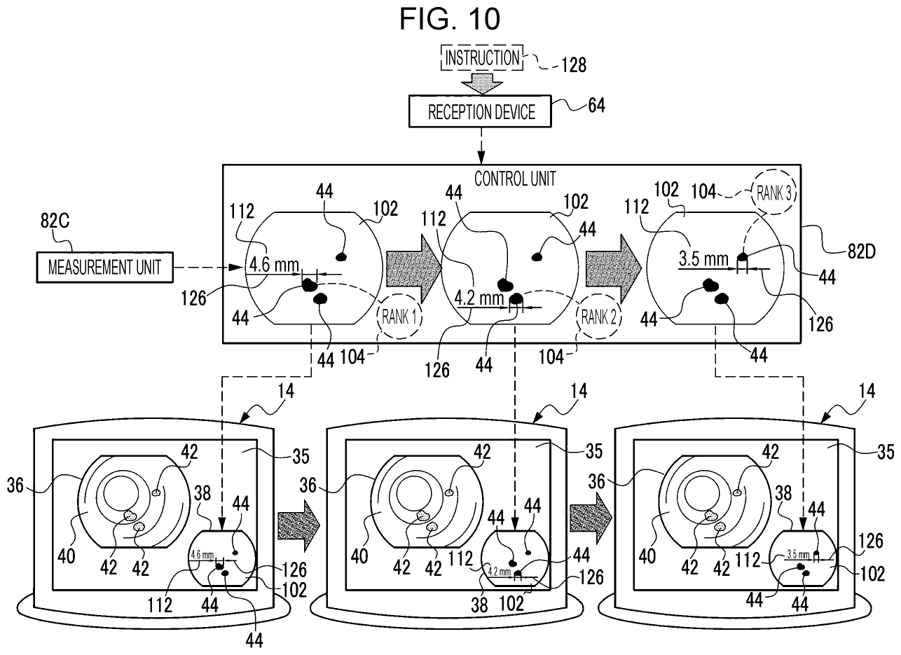

While the embodiment described above provides an example embodiment in which the sizes 112 of the plurality of lesions 42 are sequentially displayed on the screen 35 in an order based on the priority 104, the technology of the present disclosure is not limited to this. For example, as illustrated in FIG. 10, the size 112 of each of the plurality of lesions 42 may be displayed on the screen 35 (in the example illustrated in FIG. 10, in the second display region 38 on the screen 35) each time an instruction 128 is given to the endoscope 12. An example of the instruction 128 is an instruction given from the doctor 16. For example, the instruction 128 is received by the reception device 64, and the control unit 82D switches the display of the size 112 of each of the plurality of lesions 42 in an order based on the priority 104 each time the reception device 64 receives the instruction 128. Accordingly, in a case where a plurality of lesions 42 appear in the endoscopic image 40, the doctor 16 can grasp the sizes 112 of the lesions 42 sequentially, starting from the lesion 42 expected to be of highest interest to the doctor 16 to the lesion 42 expected to be of lowest interest to the doctor 16, at timings intended by the doctor 16.

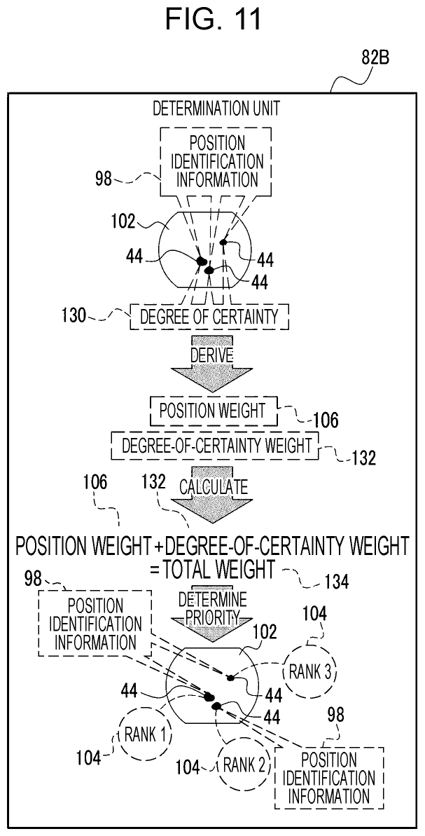

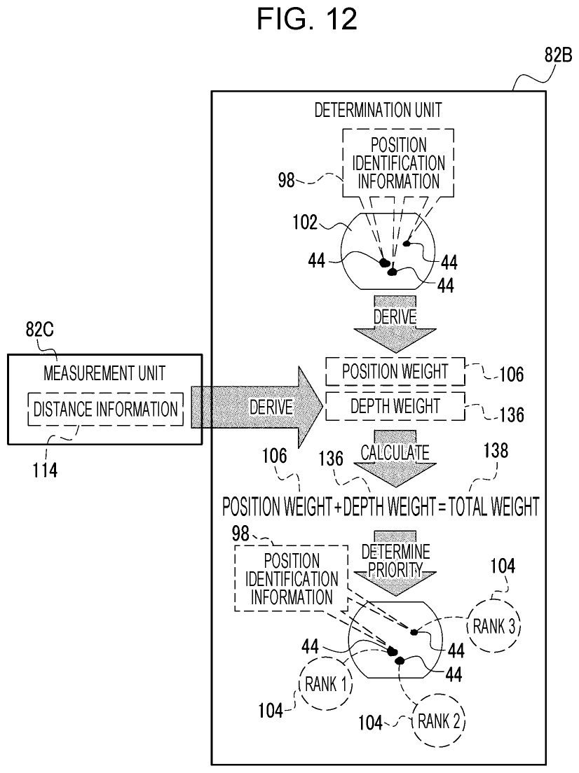

While the embodiment described above provides an example embodiment in which the priority 104 is determined based on the positions and types of the lesions 42, the technology of the present disclosure is not limited to this. For example, the priority 104 may be determined based on the positions of the lesions 42 without consideration of the types of the lesions 42. As an example, as illustrated in FIGS. 11 and 12, the priority 104 may be determined based on the position identification information 98 and information other than the type information 100.

In the example illustrated in FIG. 11, the determination unit 82B determines the priority 104 based on the position identification information 98 and a degree of certainty 130. The degree of certainty 130 is a measure of the likelihood of the position and type of each of the plurality of lesions 42, and is used for the recognition model 92 to recognize the position and type of the lesion 42. The degree of certainty 130 is obtained from the recognition model 92 for each of the plurality of lesions 42 when the positions and types of the plurality of lesions 42 are recognized in the recognition process 96 (see FIG. 5). While the measure of the likelihood of the position and type of each of the plurality of lesions 42 is illustrated as the degree of certainty 130, the technology of the present disclosure is applicable so long as the degree of certainty 130 is a measure of the likelihood of the position of each of the plurality of lesions 42.

The determination unit 82B derives a degree-of-certainty weight 132 based on the degree of certainty 130. The degree-of-certainty weight 132 increases as the degree of certainty 130 increases. For example, when the value of the degree-of-certainty weight 132 is represented by “z”, the degree-of-certainty weight 132 takes a value determined within the range of “0≤z≤0.5”. A first example of a means for deriving the degree-of-certainty weight 132 from the degree of certainty 130 is a means that uses an arithmetic expression in which the degree of certainty 130 is a dependent variable and the degree-of-certainty weight 132 is an independent variable. A second example of the means for deriving the degree-of-certainty weight 132 from the degree of certainty 130 is a means that uses a table with the degree of certainty 130 as an input and the degree-of-certainty weight 132 as an output.

The determination unit 82B calculates a total weight 134 based on the position weight 106 and the degree-of-certainty weight 132 in a manner similar to that for calculating the total weight 110 in the embodiment described above. Then, the determination unit 82B determines the priority 104 based on the total weights 134 in a manner similar to that in the embodiment described above, and assigns the determined priority 104 to the plurality of lesions 42. In the example illustrated in FIG. 11, as described above, since the degree of certainty 130 affects the determination of the priority 104, the priority 104 can be accurately determined.

While the degree-of-certainty weight 132 is illustrated in the example illustrated in FIG. 11, as an example, as illustrated in FIG. 12, a depth weight 136 may be applied instead of the degree-of-certainty weight 132.

In the example illustrated in FIG. 12, the determination unit 82B determines the priority 104 based on the position identification information 98 and the depth weight 136. The depth weight 136 is a numerical value determined in accordance with the depth from the observation position in the depth direction (hereinafter also referred to simply as “depth”). For example, the depth weight 136 is derived based on the distance information 114 extracted from the distance image 116 (see FIG. 7). The depth weight 136 may be the distance itself indicated by the distance information 114 extracted from the distance image 116, or may be a numerical value or the like obtained by dividing the distance indicated by the distance information 114 extracted from the distance image 116 into several stages to several hundreds of stages. The depth weight 136 increases as the depth decreases. This is not limiting, and the depth weight 136 may be set to increase as the depth increases. For example, whether to increase the depth weight 136 as the depth decreases or as the depth increases may be determined by an instruction or the like given from the doctor 16 through the reception device 64.

The determination unit 82B calculates a total weight 138 based on the position weight 106 and the depth weight 136 in a manner similar to that for calculating the total weight 110 in the embodiment described above. Then, the determination unit 82B determines the priority 104 based on the total weights 138 in a manner similar to that in the embodiment described above, and assigns the determined priority 104 to the plurality of lesions 42. In the example illustrated in FIG. 12, as described above, since the depth affects the determination of the priority 104, in a case where a lesion 42 located at a smaller depth is a lesion 42 of higher interest to the doctor 16 among the plurality of lesions 42 appearing in the endoscopic image 40, the doctor 16 can preferentially grasp the size 112 of the lesion 42 of high interest to the doctor 16.

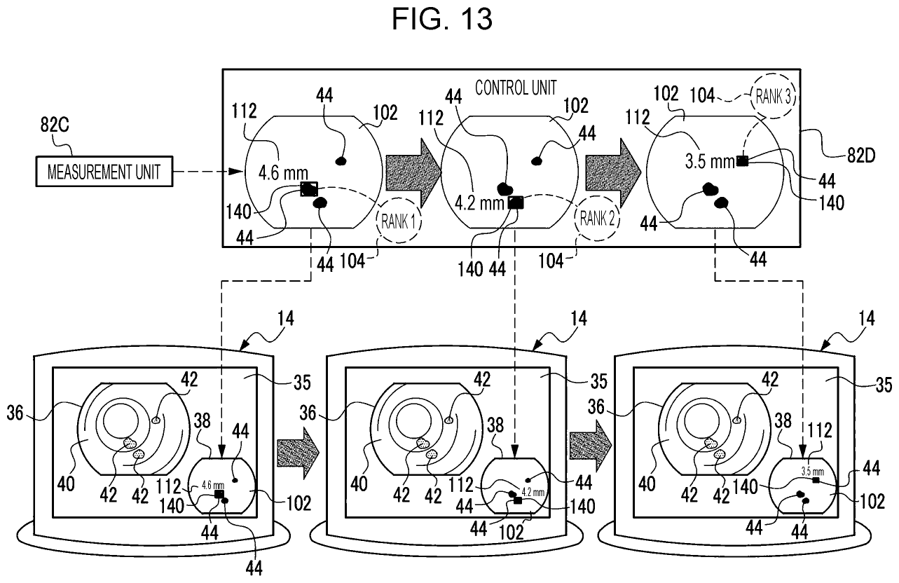

While the embodiment described above provides an example embodiment in which the dimension line 126 is displayed in association with the segmentation image 44 as information that can identify the lesion 42 corresponding to the size 112 displayed on the map 102, the technology of the present disclosure is not limited to this. For example, as illustrated in FIG. 13, a circumscribed rectangular frame 140 of the segmentation image 44 that can identify the position of the lesion 42 corresponding to the size 112 displayed on the map 102 in the endoscopic image 40 may be displayed on the map 102. Also in this case, the dimension line 126 may be displayed on the map 102 together with the circumscribed rectangular frame 140. For example, when the positions of the lesions 42 are recognized by using AI in a bounding box method, a bounding box may be used as the circumscribed rectangular frame 140. The circumscribed rectangular frame 140 is an example of “region identification information” according to the technology of the present disclosure.

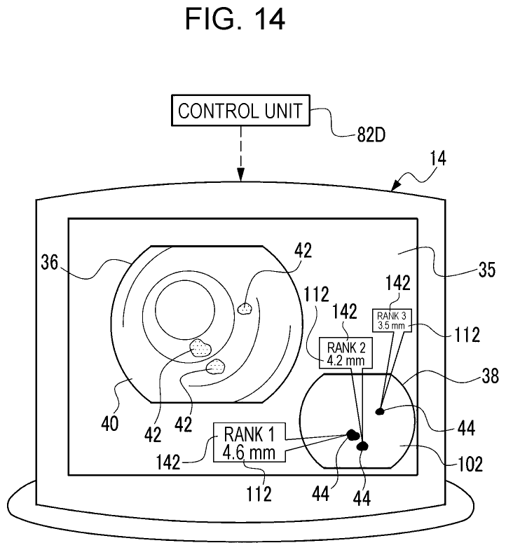

While the embodiment described above provides an example embodiment in which the sizes 112 are sequentially displayed on the map 102 in an order based on the priority 104, this is merely an example. For example, as illustrated in FIG. 14, the sizes 112 may be displayed outside the map 102 (that is, outside the second display region 38). In the example illustrated in FIG. 14, the sizes 112 are displayed outside the map 102 in a pop-up manner from within the map 102. In the example illustrated in FIG. 14, a text box appears from each of the plurality of segmentation images 44, and each text box includes text information 142 that can identify the size 112 and the priority 104. This allows the doctor 16 to simultaneously view the sizes 112 and the priority 104 of the plurality of lesions 42 on the screen 35. In the example illustrated in FIG. 14, the text information 142 is presented, but this is merely an example, and information (for example, an image, a symbol, or the like) that allows the doctor 16 to visually recognize the priority 104 may be used.

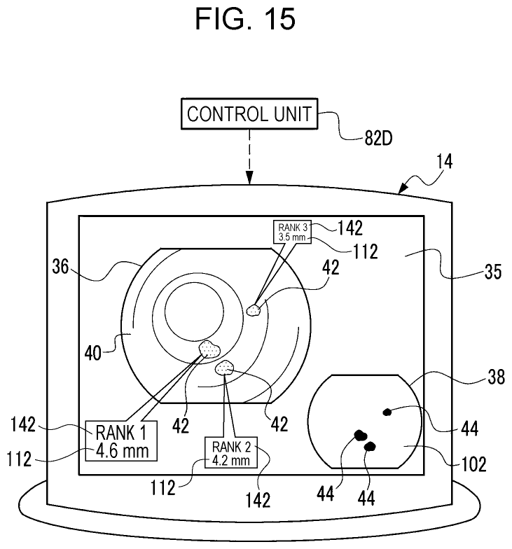

While the example illustrated in FIG. 14 provides an example embodiment in which the size 112 and the text information 142 are displayed in a pop-up manner from each of the plurality of segmentation images 44, this is merely an example. For example, as illustrated in FIG. 15, the size 112 and the text information 142 may be displayed in a pop-up manner from each of the plurality of lesions 42 appearing in the endoscopic image 40 in a manner similar to that in which the size 112 and the text information 142 are displayed in a pop-up manner from each of the plurality of segmentation images 44.

In the example illustrated in FIG. 15, the sizes 112 and the text information 142 are displayed in display sizes based on the priority 104. For example, the size 112 and the text information 142 for which the priority 104 is higher are displayed in a larger display size. This is merely an example, and it is sufficient that the size 112 and the text information 142 for which the priority 104 is higher be displayed in a more highlighted manner.

As described above, the sizes 112 are displayed on the screen 35 in a display format based on the priority 104. Accordingly, in a case where a plurality of lesions 42 appear in the endoscopic image 40, the doctor 16 can visually identify the size 112 of the lesion 42 expected to be of high interest to the doctor 16 and the size 112 of the lesion 42 expected to be of low interest to the doctor 16.

In the examples illustrated in FIGS. 14 and 15, a pop-up display using a text box is illustrated. However, this is merely an example, and it is sufficient that the size 112 be displayed on the screen 35 in a display format that can identify which lesion 42 the size 112 corresponds to (for example, a display format in which the segmentation image 44 on the map 102 or the lesion 42 in the endoscopic image 40 and the size 112 are connected by a line). Likewise, it is sufficient that the size 112 of the text information 142 be displayed on the screen 35 in a display format that can identify which lesion 42 the text information 142 is related to.

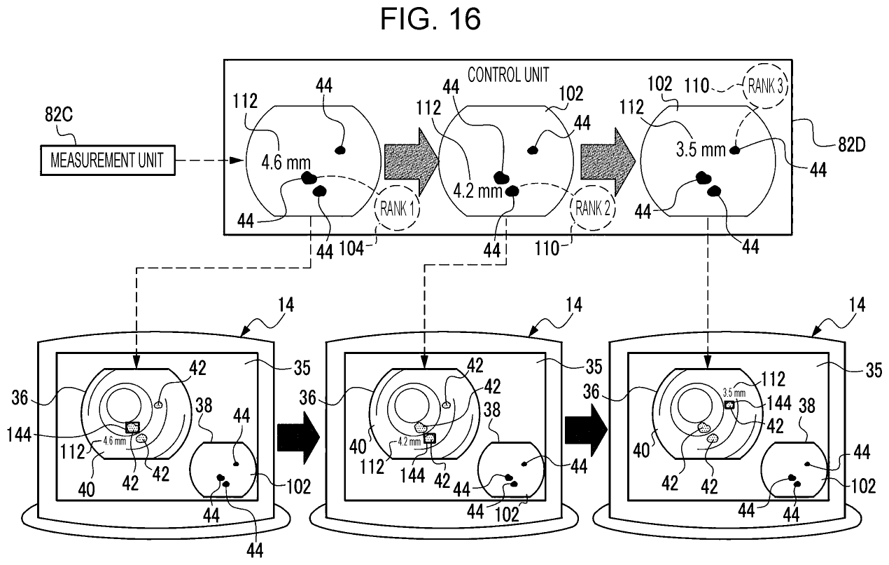

While the embodiment described above provides an example embodiment in which the size 112 of each of the plurality of lesions 42 is displayed on the map 102, the technology of the present disclosure is not limited to this. For example, as illustrated in FIG. 16, a size 112 may be displayed in the endoscopic image 40. This allows the doctor 16 to visually recognize the size 112 of each of the plurality of lesions 42 together with the endoscopic image 40.

The size 112 may be displayed in a display format in which alpha blending has been performed. Alternatively, the size 112 may be displayed in a display format (for example, font size, font color, transparency, and/or the like) that can identify the priority 104.

As an example, as illustrated in FIG. 16, a circumscribed rectangular frame 144 of the lesion 42 corresponding to the size 112 displayed in the endoscopic image 40 may be displayed in the endoscopic image 40. For example, when the positions of the lesions 42 are recognized by using AI in a bounding box method, a bounding box may be used as the circumscribed rectangular frame 144.

In the example illustrated in FIG. 16, the display of the circumscribed rectangular frame 144 is switched each time the display of the size 112 is switched in accordance with the priority 104 so that which lesion 42 the size 112 displayed in the endoscopic image 40 corresponds to can be identified. The circumscribed rectangular frame 144 is an example of “region identification information” according to the technology of the present disclosure.

As described above, the circumscribed rectangular frame 144 that encloses the lesion 42 corresponding to the size 112 displayed in the endoscopic image 40 is displayed in the endoscopic image 40. This allows the doctor 16 to visually and easily recognize which lesion 42 the size 112 displayed in the endoscopic image 40 corresponds to among the plurality of lesions 42 appearing in the endoscopic image 40.

While the example illustrated in FIG. 16 illustrates an example embodiment in which the display of the size 112 and the display of the circumscribed rectangular frame 144 in the endoscopic image 40 are switched in an order based on the priority 104, the sizes 112 of the plurality of lesions 42 may be collectively displayed in the endoscopic image 40. In this case, assigning a dimension line to each of the plurality of lesions 42 makes it possible to identify which lesion 42 each of the plurality of displayed sizes 112 corresponds to. Further, the display format such as the line type, color, and/or luminance of the circumscribed rectangular frame 144 may be changed in accordance with the priority 104.

While each of the examples described above provides an example embodiment in which the size 112 is displayed on the screen 35, this is merely an example. The size 112 may be displayed on the screen 35 and/or at least one screen other than the screen 35. In addition to this, the screen 35 and/or at least one screen other than the screen 35 may display information that can identify the priority 104, or may display information that can identify the lesion 42 corresponding to the displayed size 112 (for example, a dimension line, a circumscribed rectangular frame, and/or the like).

While each of the examples described above provides an example embodiment in which the priority 104 is determined based on two pieces of information, namely, the position identification information 98 and information other than the position identification information 98, the priority 104 may be determined based on three or more pieces of information including the position identification information 98 (for example, three or more pieces of information among the position identification information 98, the type information 100, the degree of certainty 130, and the depth).

The priority 104 may be determined based on one or more pieces of information (for example, one or more pieces of information among the type information 100, the degree of certainty 130, and the depth) other than the position identification information 98.

While the embodiment described above provides an example embodiment in which the sizes 112 are measured on a frame-by-frame basis, this is merely an example. The statistical values (for example, mean values, median values, mode values, or the like) of the sizes 112 measured for endoscopic images 40 of a plurality of frames along a time series may be displayed in a display format similar to that in the embodiment described above.

For example, the sizes 112 may be measured when the amounts of shift in the positions of the lesions 42 between a plurality of frames are less than a threshold value, and the measured sizes 112 themselves or the statistical values of the sizes 112 measured for endoscopic images 40 of a plurality of frames along a time series may be displayed on the screen 35.

While the embodiment described above describes an example embodiment in which the positions of the lesions 42 are recognized for each endoscopic image 40 by using an AI-based segmentation method, the technology of the present disclosure is not limited to this. For example, the positions of the lesions 42 may be recognized for each endoscopic image 40 by using an AI-based bounding box method.

In this case, it is sufficient that the amount of change of the bounding box be calculated by the processor 82 and it be determined whether to measure the size 112 of the lesion 42, based on the amount of change of the bounding box in a manner similar to that in the embodiment described above.

The amount of change of the bounding box means, for example, the amount of change in the position of the lesion 42. The amount of change in the position of the lesion 42 may be the amount of change in the position of the lesion 42 between adjacent endoscopic images 40 along the time series, or the amount of change in the position of the lesion 42 between three or more frames of endoscopic images 40 along the time series (for example, the statistical value such as the mean value, the median value, the mode value, or the maximum value of the amounts of change between three or more frames of endoscopic images 40 along the time series). Alternatively, the amount of change in the position of the lesion 42 may be the amount of change in the position of the lesion 42 between a plurality of frames along a time series with an interval of one or more frames therebetween.

In the embodiment described above, the AI-based object recognition process is presented as an example of the recognition process 96. However, the technology of the present disclosure is not limited to this. The recognition unit 82A may recognize the lesions 42 appearing in the endoscopic image 40 in response to the execution of a non-AI-based object recognition process (for example, template matching or the like).

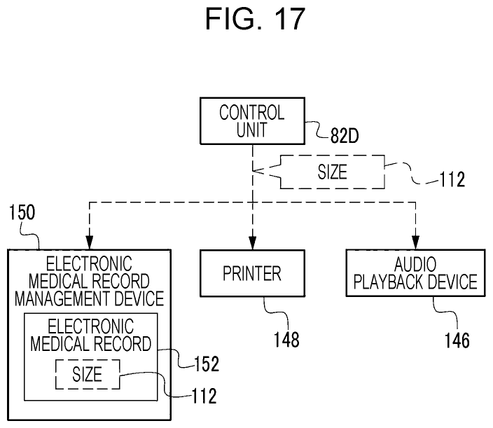

In the embodiment described above, the sizes 112 are output to the display device 14, by way of example. However, the technology of the present disclosure is not limited to this, and the sizes 112 may be output to a destination other than the display device 14. As an example, as illustrated in FIG. 17, the sizes 112 may be output to an audio playback device 146, a printer 148, an electronic medical record management device 150, and/or the like as a destination.

The sizes 112 may be output as audio by the audio playback device 146. The sizes 112 may be printed as texts or the like on a medium (for example, a sheet) or the like by the printer 148. The sizes 112 may be stored in an electronic medical record 152 managed by the electronic medical record management device 150.

While the embodiment described above describes an example embodiment in which the arithmetic expression 124 is used to calculate the sizes 112, the technology of the present disclosure is not limited to this. The sizes 112 may be measured by performing an AI-based process on the endoscopic image 40. In this case, for example, a trained model is used that, in response to an input of the endoscopic image 40 including the lesions 42, outputs the sizes 112 of the lesions 42. To generate the trained model, deep learning is performed on a neural network by using training data in which lesions appearing in images used as example data are assigned annotations indicating the sizes of the lesions as ground-truth data.

While the embodiment described above describes an example embodiment in which the distance information 114 is derived using the distance derivation model 94, the technology of the present disclosure is not limited to this. Other methods for deriving the distance information 114 using an AI-based method include, for example, a method for combining segmentation and depth estimation (for example, regression learning to provide the distance information 114 to the entire image (for example, all the pixels constituting the image) or unsupervised learning to learn the distance of the entire image in an unsupervised way).

While the embodiment described above provides an example embodiment in which the distance from the camera 52 to the intestinal wall 24 is derived by using an AI-based method, the distance from the camera 52 to the intestinal wall 24 may be actually measured. In this case, for example, the tip portion 50 (see FIG. 2) may be provided with a distance-measuring sensor, and the distance from the camera 52 to the intestinal wall 24 may be measured by the distance-measuring sensor.

While the endoscopic image 40 is illustrated in the embodiment described above, the technology of the present disclosure is not limited to this. The technology of the present disclosure is also applicable to a medical image (for example, an image obtained by a modality other than the endoscope 12, such as a radiographic image or an ultrasound image) other than the endoscopic image 40.

While the embodiment described above provides an example embodiment in which the sizes 112 of the lesions 42 appearing in a moving image are measured, this is merely an example. The technology of the present disclosure is also applicable to a stop-motion image or a still image in which the lesions 42 appear.

While the embodiment described above provides an example embodiment in which the distance information 114 extracted from the distance image 116 is input to the arithmetic expression 124, the technology of the present disclosure is not limited to this.

For example, it is sufficient that the distance information 98 corresponding to the position identified from the position identification information 98 be extracted from among all the pieces of distance information 114 output from the distance derivation model 94, without the distance image 116 being generated, and the extracted distance information 114 be input to the arithmetic expression 124.

While the embodiment described above provides an example embodiment in which the medical support process is performed by the processor 82 of the computer 78 included in the endoscope 12, the technology of the present disclosure is not limited to this, and a device external to the endoscope 12 may perform the medical support process. Examples of the device external to the endoscope 12 include at least one server and/or at least one personal computer connected to the endoscope 12 in a communicable manner. Alternatively, the medical support process may be performed by a plurality of devices in a distributed manner.

While the embodiment described above provides an example embodiment in which the medical support program 90 is stored in the NVM 86, the technology of the present disclosure is not limited to this. For example, the medical support program 90 may be stored in a portable non-transitory computer-readable storage medium such as an SSD or a USB memory. The medical support program 90 stored in the non-transitory storage medium is installed in the computer 78 of the endoscope 12. The processor 82 executes the medical support process in accordance with the medical support program 90.

Alternatively, the medical support program 90 may be stored in a storage device of another computer, a server, or the like connected to the endoscope 12 via a network, and the medical support program 90 may be downloaded in response to a request from the endoscope 12 and installed in the computer 78.

Not all, but a portion, of the medical support program 90 may be stored in a storage device of another computer, a server device, or the like connected to the endoscope 12, or not all, but a portion, of the medical support program 90 may be stored in the NVM 86.

Examples of a hardware resource that executes the medical support process may include the following various processors. The processors include, for example, a CPU that is a general-purpose processor configured to execute software, that is, a program, to function as a hardware resource that executes the medical support process. The processors further include, for example, a dedicated electric circuit that is a processor having a circuit configuration designed specifically for executing specific processing, such as an FPGA, a PLD, or an ASIC. Each of the processors incorporates or is connected to a memory, and uses the memory to execute the medical support process.

The hardware resource that executes the medical support process may be configured as one of the various processors or as a combination of two or more processors of the same type or different types (for example, a combination of a plurality of FPGAs or a combination of a CPU and an FPGA). The hardware resource that executes the medical support process may be a single processor.

Examples of configuring the hardware resource as a single processor include, first, a form in which a single processor is configured as a combination of one or more CPUs and software and the processor functions as a hardware resource that executes the medical support process. The examples include, second, a form in which, as typified by an SoC or the like, a processor is used in which the functions of the entire system including a plurality of hardware resources that execute the medical support process are implemented as one IC chip. As described above, the medical support process is implemented by using one or more of the various processors described above as hardware resources.

More specifically, the hardware structure of these various processors may be an electric circuit in which circuit elements such as semiconductor elements are combined. The medical support process described above is merely an example.

Thus, it goes without saying that unnecessary steps may be deleted, new steps may be added, or the processing order may be changed without departing from the gist.

The description and drawings presented above provide detailed descriptions of portions according to the technology of the present disclosure and are merely examples of the technology of the present disclosure. For example, the descriptions related to the configurations, functions, operations, and effects described above are descriptions related to an example of the configurations, functions, operations, and effects of portions according to the technology of the present disclosure. Thus, it goes without saying that unnecessary portions may be deleted or new elements may be added or substituted in the description and drawings presented above without departing from the gist of the technology of the present disclosure. To avoid complexity and facilitate understanding of portions according to the technology of the present disclosure, descriptions related to common general technical knowledge and the like, for which no specific explanation is required to implement the technology of the present disclosure, are omitted in the description and drawings presented above.

As used herein, “A and/or B” is synonymous with “at least one of A or B”. That is, “A and/or B” means only A, only B, or a combination of A and B. In this specification, furthermore, a concept similar to that of “A and/or B” is applied also to the expression of three or more matters in combination with “and/or”.

All publications, patent applications, and technical standards described herein are incorporated herein by reference to the same extent as if each individual publication, patent application, and technical standard were specifically and individually indicated to be incorporated by reference.

In relation to the embodiment described above, the following appendices are further disclosed.

### APPENDIX 1

A medical support device including:
a processor,
the processor being configured to:
recognize, based on a medical image in which a plurality of observation target regions appear, positions of the plurality of observation target regions in the medical image;
determine a priority of the plurality of observation target regions, based on the positions; and
measure sizes of the plurality of observation target regions in accordance with the priority.

### APPENDIX 2

An endoscope including:
the medical support device according to Appendix 1; and
a module to be inserted into a body including the observation target regions to acquire the medical image by imaging the observation target regions.

### APPENDIX 3

A medical support method including:
recognizing, based on a medical image in which a plurality of observation target regions appear, positions of the plurality of observation target regions in the medical image;
determining a priority of the plurality of observation target regions, based on the positions; and
measuring sizes of the plurality of observation target regions in accordance with the priority.

### APPENDIX 4

A program for causing a computer to execute a medical support process including:
recognizing, based on a medical image in which a plurality of observation target regions appear, positions of the plurality of observation target regions in the medical image;
determining a priority of the plurality of observation target regions, based on the positions; and
measuring sizes of the plurality of observation target regions in accordance with the priority.

What is claimed is:

1. A medical support device comprising:
a processor,
the processor being configured to:
recognize, based on a medical image in which a plurality of observation target regions appear, positions of the plurality of observation target regions in the medical image;
determine a priority of the plurality of observation target regions, based on the positions;
measure sizes of the plurality of observation target regions; and
output the sizes in accordance with the priority.

2. The medical support device according to claim 1, wherein
the processor is configured to output the sizes of the observation target regions one at a time in an order based on the priority.

3. The medical support device according to claim 2, wherein
each of the sizes of the observation target regions is output each time an instruction is given.

4. The medical support device according to claim 1, wherein
the sizes are output by displaying the sizes on a screen.

5. The medical support device according to claim 4, wherein
the sizes are displayed on the screen in a display format corresponding to the priority.

6. The medical support device according to claim 4, wherein
the screen displays the medical image, and
the sizes are displayed in the medical image.

7. The medical support device according to claim 4, wherein
the screen displays the medical image, and region identification information is displayed in the medical image, the region identification information being information that allows an observation target region corresponding to a size that has been output among the sizes to be identified from among the observation target regions.

8. The medical support device according to claim 4, wherein
the screen includes a first display region and a second display region,
the first display region displays the medical image, and
the second display region displays a map indicating a distribution of the positions of the observation target regions, and region identification information is displayed on the map, the region identification information being information that allows an observation target region corresponding to a size that has been output among the sizes to be identified from among the observation target regions.

9. The medical support device according to claim 4, wherein
a size to be displayed on the screen among the sizes is switched in accordance with the priority.

10. The medical support device according to claim 9, wherein
the size to be displayed on the screen is switched each time an instruction is given.

11. The medical support device according to claim 1, wherein
the positions are recognized by a method using AI, and
the priority is determined based on a degree of certainty obtained from the AI.

12. The medical support device according to claim 1, wherein
the priority is higher for a position closer to a center of the medical image among the positions.

13. The medical support device according to claim 1, wherein
the processor is configured to acquire depths of the plurality of observation target regions, and
the priority is determined based on the positions and the depths.

14. The medical support device according to claim 1, wherein
the processor is configured to recognize types of the observation target regions, based on the medical image, and
the priority is determined based on the positions and the types.

15. The medical support device according to claim 1, wherein
the processor is configured to measure the sizes in accordance with the priority.

16. The medical support device according to claim 1, wherein
the medical image is an endoscopic image obtained by imaging with an endoscope.

17. The medical support device according to claim 1, wherein
the observation target regions are lesions.

18. An endoscope comprising:
the medical support device according to claim 1; and
a module to be inserted into a body including the observation target regions to acquire the medical image by imaging the observation target regions.

19. A medical support method comprising:
recognizing, based on a medical image in which a plurality of observation target regions appear, positions of the plurality of observation target regions in the medical image;
determining a priority of the plurality of observation target regions, based on the positions;
measuring sizes of the plurality of observation target regions; and
outputting the sizes in accordance with the priority.

20. A non-transitory computer-readable storage medium storing a program executable by a computer to execute a medical support process, the medical support process comprising:
recognizing, based on a medical image in which a plurality of observation target regions appear, positions of the plurality of observation target regions in the medical image;
determining a priority of the plurality of observation target regions, based on the positions;
measuring sizes of the plurality of observation target regions; and
outputting the sizes in accordance with the priority.
